(19) United States
(12) Patent Application Publication
(10) Pub. No.: US 2010/0324594 A1
(43) Pub. Date: Dec. 23, 2010

(54) DEVICE FOR COSMETIC TREATMENT OF HAIRY BODY PARTS

(76) Inventor: Serena Mercanti, Ancona (IT)

Correspondence Address: HEDMAN & COSTIGAN, P.C., 1230 AVENUE OF THE AMERICAS, 7th floor, NEW YORK, NY, 10020, US

(21) Appl. No.: 12/733,956

(22) PCT Filed: May 20, 2008
(86) PCT No.: PCT/IT2008/000337

§ 371 Date: Aug. 2, 2010

(30) Foreign Application Priority Data
Oct. 4, 2007 (IT) AN2007U000022
Dec. 6, 2007 (IT) AN2007A000065

(51) Int. Cl. A61B 17/50

(52) U.S. Cl. 606/211

(57) ABSTRACT

The present invention relates to a device (1) for cosmetic treatment of hairy body parts, such as eyebrows, comprising a pair of tweezers (2), having a couple of flexible arms (2′, 2″) connected to each other at an end; characterised in that said device (1) further comprises at least one comb/brush member (3) each coupled to one of said arms (2′).

This invention concerns a device for cosmetic treatment of hairy body parts.

More specifically, the invention relates to a device for the treatment of hairs, such as eyebrows or surface hair and like, comprising a pair of tweezers, which allows to comb and direct the hair to be removed or treated.

As it is well known, at present various kinds of devices or tools for skin cosmetic treatment are available on the market. In particular, a widely used tool is the tweezers, used for handling small things such as hairs.

At present, a beautician has to use a brush or a comb to select and direct, for example, an eyebrow to be removed and then take up a pair of tweezers to remove it. Nevertheless, there is often the drawback that in the meantime, while the beautician takes up said tweezers, the eyebrow can move and the beautician has to select it again by the comb, with a waste of time.

Thus, the difficulty of the actual solutions is quite evident.

Examples of device for cosmetic treatment of hairy body parts are known from French patent application FR 2887128 A and United States patent application US 2006028040, which relate to ordinary cosmetic tweezers for eyebrows, provided with flexible metal arms. Both models of tweezers from the aforementioned publications do not allow to comb the hair to be removed in an efficient, effective and ergonomic way for the user, for said hair to be then easily handled, removed or treated.

In view of the above, it is an object of the present invention to provide a device having the function of both treating and handling hairs, which can be used with a natural non-stressful (ergonomic) movement and provide the best direction of the hair to be removed or to be treated.

It is a further object of the present invention to provide a modular device, so that its parts can be easily interchangeable, in order to allow a high level of hygiene, for it to be profitably used even by professional beauticians.

It is specific subject-matter of this invention a device for cosmetic treatment of hairy body parts, such as eyebrows, comprising a pair of tweezers, having a couple of flexible arms connected to each other at an end; characterised in that said device further comprises at least one comb/brush member each coupled to one of said arms.

Still according to the invention, said device could comprise two comb/brush members, each comb/brush member (3) being coupled to one of said arms.

Additionally according to the invention, said at least one comb/brush member could be fixed at the free end portion of the respective arm.

Further according to the invention, said at least one comb/brush member could be coupled to the respective arm by screw and/or glue and/or a hook and or a clip and/or a notch.

Still according to the invention, said at least one comb/brush member could be movably coupled to the respective arm.

Additionally according to the invention, said at least one comb/brush member could be slidably coupled to the respective arm.

Further according to the invention, said at least one comb/brush member could comprise a projection, and the respective arm could have a guide groove into which said projection of said comb/brush member is insertable, in order for it to slide along said guide groove, thereby permitting its positioning.

Still according to the invention, said projection and the respective arm could be coupled by magnetic force.

Additionally according to the invention, said comb/brush member could have a two or more coupling studs, and the respective arm could have a plurality of holes along its length, in which said coupling studs of said comb/brush member are insertable and removably coupled.

Further according to the invention, said pair of tweezers could be made of metal, polymeric or composite material, such as carbon fibre or similar material.

Still according to the invention, said comb/brush member could be made of plastic material, rubber, natural or synthetic bristles.

Preferably according to the invention, the comb/brush member could be changed or replaced so as to be disposable.

This invention will be now described, by way of explanation and not by way of limitation, according to its preferred embodiments, by particularly referring to the attached drawings, in which:

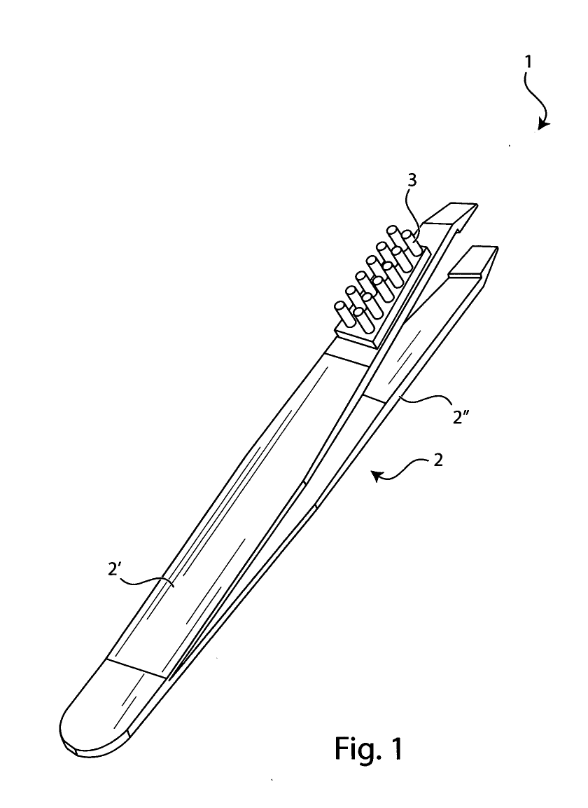

FIG. 1 shows a perspective view of a first embodiment of the device for cosmetic treatment of hairy body parts according to the present invention;

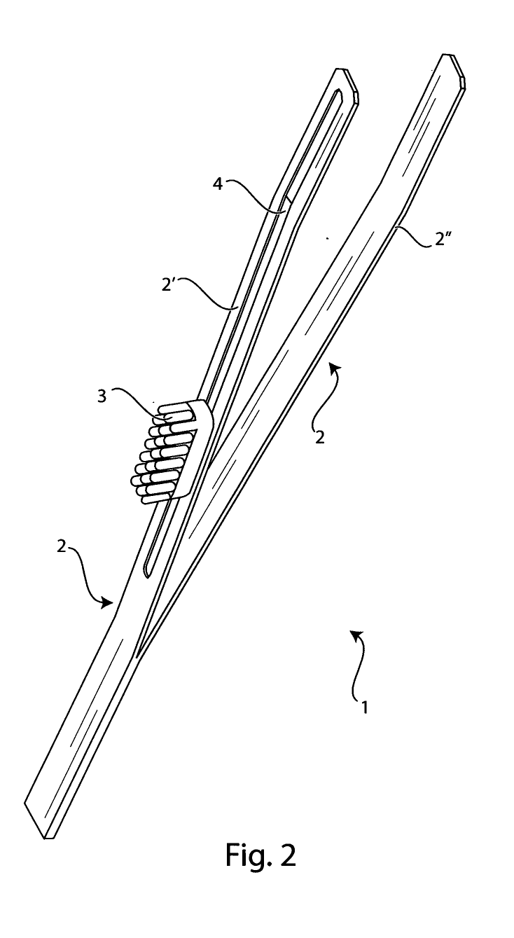

FIG. 2 shows a perspective view of a second embodiment of the device for cosmetic treatment of hairy body parts according to the present invention;

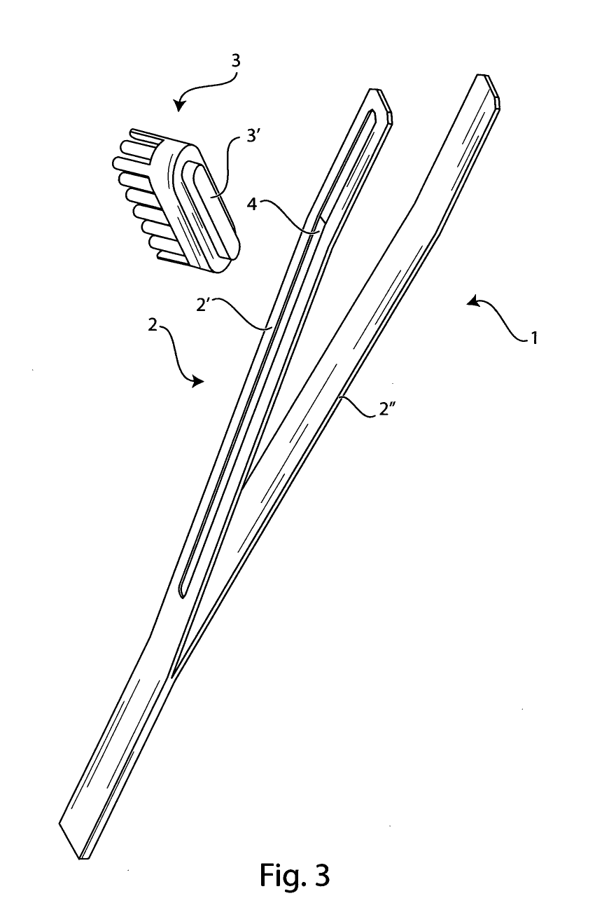

FIG. 3 shows the device according to FIG. 2 in which a brush/comb member is uncoupled;

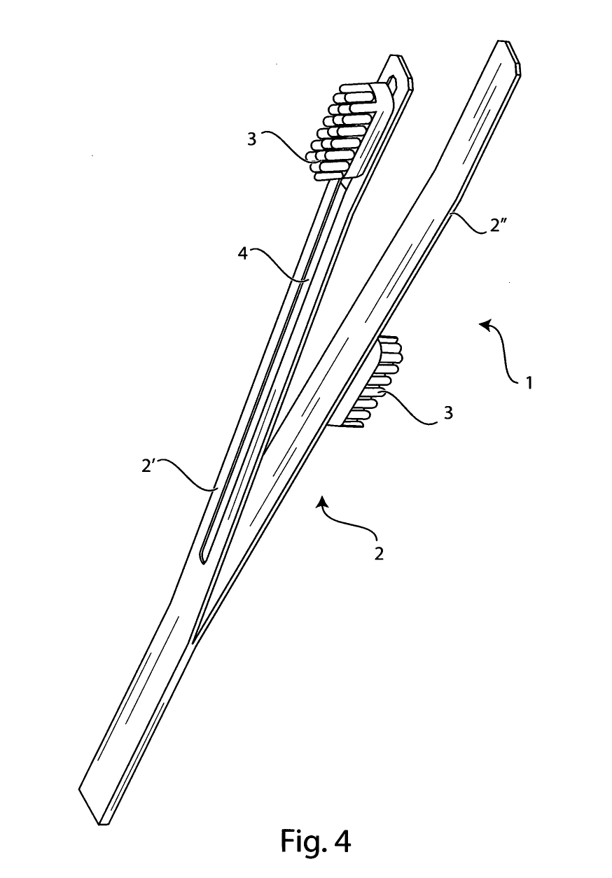

FIG. 4 shows a perspective view of a third embodiment of the device for cosmetic treatment of hairy body parts according to the present invention; and

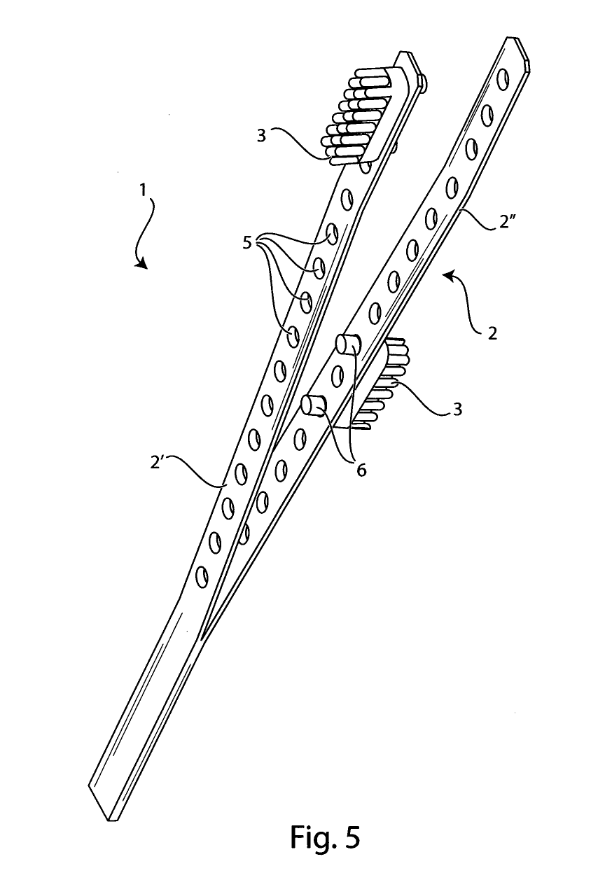

FIG. 5 shows a perspective view of a fourth embodiment of the device for cosmetic treatment of hairy body parts according to the present invention.

Referring to FIG. 1, a first embodiment of a device 1 for cosmetic treatment of hairy body parts is shown, in order to outline basic operation mode, as it will be analysed in the following.

Said device 1 comprises essentially a pair of tweezers 2 made by a first and a second deformed flexible arm 2′ and 2″, mutually joined at their ends. The joining can be made by different ways or technologies according to the type of the used material.

Said device 1 comprises further a comb/brush member 3 coupled to one of said arms, i.e. in this case said first arm 2′, at the second end portion thereof.

A user can select, comb and/or direct a hair by the comb/brush member 3 to simplify the following removal by means of the tweezers 2.

For example, it is possible to comb the eyebrows during depilation, thereby assuring a high comfort of the operation, improving the accuracy and reducing the working time.

Furthermore, the comb/brush member 3 allows to comb the eyebrows again after removing one hair, with a rapid, natural and ergonomic movement, in order to identify, isolate and rapidly remove a further hair.

In this embodiment, the comb/brush member 3 can be coupled to said first arm 2′ e.g. by screws, glue, a hook, a clip or a notch. Advantageously, the comb/brush member 3 can be even integrated into the respective arm, e.g. by moulding.

FIGS. 2 and 3 show a second embodiment of the device 1 for cosmetic treatment according to this invention. The comb/brush member 3 is slidably coupled to the first arm 2′ by a guide groove 4. More in detail, the comb/brush member 3 comprises at a rear projection 3′ insertable in the guide groove 4 of the associated arm 2′. Therefore, the comb/brush member 3 can be shifted and placed in different positions along the guide groove 4 of said arm 2′, as desired by the user.

Advantageously, said projection 3′ is made by a magnetic material, for it to be coupled to the metallic arm 2′ in the desired position during operation.

The teeth or bristles of the comb/brush 2 are preferably made of suitable soft materials and are shaped so as to avoid sharp corners, in such a way as not to scratch the eyebrow skin during use. Therefore, they can be made of plastic material, rubber, wood or natural or synthetic bristles.

The teeth or bristles have an arbitrary section, with deeper extension than the height of the eyebrows (in a perpendicular direction to the skin) and are provided in suitable number to treat at least the width of the eyebrows (smaller dimension of the eyebrow parallel to the skin) with one movement.

FIG. 4 shows a further embodiment of the invention, in which the device 1 comprises two different comb/brush members 3, each slidably coupled to a respective arm 2′ or 2″. Furthermore, each comb/brush member can be different from the other, in order to provide the user with two different kinds of brushes or combs integrated in the same tool, each one of which can be easily and independently positioned.

FIG. 5 discloses a fourth embodiment of the device 1, which comprises two different comb/brush members 3, coupled respectively to the arms 2′ and 2″. In this case, said arms have a plurality of holes 5 along their length, while each comb/brush member 3 is provided with two or more coupling studs 6, to be inserted in said holes 5. Then, this embodiment provides for coupling surfaces and perpendicular to the larger dimension of the sides of the tweezers rather than parallel.

When a displacement of the comb/brush member 3 is needed, in order to change the position of said comb/brush member 3 along the respective arm, a user can decouple said comb/brush member 3, by extracting said coupling studs 6 from said holes 5, and insert the same in other holes 5, where desired. Hence in this case, it is necessary to dismount and mount the comb/brush member 3 instead of simply sliding it.

As it can be easily seen, the device has a limited number of discrete engagement positions of the comb/brush member 3 to the device 1.

Lastly, the comb/brush member 3 can be advantageously obtained on an insert that is reversibly applied to the arm of the device 1. Said insert can be indifferently applied with prismatic guides, snapped in, attached or fitted with screws.

The use of two comb/brush members (one on each arm of the tweezers) improves the efficiency of the invention, because it can be indifferently used by right-handed or left-handed users.

A number of modifications can be made to the present invention by that expert of the art, who can choose the most suitable dimensions and materials according to the specific use.

An advantage of the present invention is that the device is suitable both for professional and personal use.

A further advantage of the invention is that the comb/brush member can be disposable.

This invention have been above described by way of illustration, but not by way of limitation according to its preferred embodiments and it should be understood that those skilled in the art can make other modifications and changes without departing from the scope of the invention as defined in the following claims.

1. A device (1) for cosmetic treatment of hairy body parts, such as eyebrows, comprising a pair of tweezers (2), having a couple of flexible arms (2′, 2″) connected to each other at an end; characterised in that said device (1) further comprises at least one comb/brush member (3) each coupled to one of said arms (2′).

2. A device (1) according to claim 1, characterised in that it comprises two comb/brush members (3), each comb/brush member (3) being coupled to one of said arms (2′, 2″).

3. A device (1) according to claim 1, characterised in that said at least one comb/brush member (3) is fixed at the free end portion of the respective arm.

4. A device (1) according to claim 1, characterised in that said at least one comb/brush member (3) is coupled to the respective arm (2′) by screw and/or glue and/or a hook and or a clip and/or a notch.

5. A device (1) according to claim 1, characterised in that said at least one comb/brush member (3) is movably coupled to the respective arm (2′).

6. A device (1) according to claim 5, characterised in that said at least one comb/brush member (3) is slidably coupled to the respective arm (2′).

7. A device (1) according to claim 6, characterised in that said at least one comb/brush member (3) comprises a projection (3′), and the respective arm (2′) has a guide groove (4) into which said projection (3′) of said comb/brush member (3) is insertable, in order for it to slide along said guide groove (4), thereby permitting its positioning.

8. A device (1) according to claim 7, characterised in that said projection (3′) and the respective arm (2′) are coupled by magnetic force.

9. A device (1) according to claim 1, characterised in that said comb/brush member (3) has two or more coupling studs (6), and the respective arm (2′) has a plurality of holes (5) along its length, in which said coupling studs (6) of said comb/brush member (3) are insertable and removably coupled.

10. A device (1) according to claim 1, characterised in that said pair of tweezers (2) are made of metal, polymeric or composite material, such as carbon fibre or similar material.

11. A device (1) according to claim 1, characterised in that said comb/brush member (3) is made of plastic material, rubber, natural or synthetic bristles.

12. A device (1) according to claim 1, characterised in that the comb/brush member (2) can be changed or replaced so as to be disposable.
